(19) United States
(12) Patent Application Publication
(10) Pub. No.: US 2008/0254551 A1
(43) Pub. Date: Oct. 16, 2008

(54) Immunoprecipitaion-Based Method to Purify and Characterise Biological Macromolecular Complexes

(76) Inventor: Achsel Tilmann, Grottaferrata (IT)

Correspondence Address: Breiner & Breiner, L.L.C., P.O. Box 19290, Alexandria, VA, 22320-0290, US

(21) Appl. No.: 11/631,154

(22) PCT Filed: Dec. 30, 2004
(86) PCT No.: PCT/IT04/00738

§ 371 Date: Dec. 29, 2006

(51) Int. Cl. G01N 33/543; C07K 1/00; C12N 11/00; C07K 14/00; C07H 21/00; C12N 5/06; C12N 1/20

(52) U.S. Cl. 436/518; 530/413; 435/174; 530/350; 536/23.1; 435/325; 435/252.3

(57) ABSTRACT

This invention concerns an artificial adapter protein that combines an antibody-binding activity with two affinity tags and its use in isolation of antibody-antigen complexes. Using this adapter protein, complexes can be obtained at good yield and in the high purity necessary for the identification of all biological macromolecules that are associated with the antigen.

### TECHNICAL FIELD

This invention, concerns an artificial adapter protein that combines an antibody-binding activity with two affinity tags and its use in isolation of antibody-antigen complexes. Using this adapter protein, complexes can be obtained at good yield and in the high purity necessary for the identification of all biological macromolecules that are associated with the antigen.

### DESCRIPTION OF THE RELATED ART

In the post-genomic era, all human proteins are known, at least by their sequence. Now it becomes important to understand their function, and thus the focus of research has shifted towards functional characterisation of the proteins. One important clue to the function of a given protein comes from the identification of biological macromolecules—proteins, nucleic acids, or other—that associate with the target protein and thus form a functional unit. Knowledge of these macromolecules is also of considerable interest in pharmaceutical research, as every interaction with a protein of interest is a potential drug target.

Therefore, methods to identify protein interactions have received considerable attention, and at least two approaches have been tried in order to develop a simple method that can identify and characterise interactions of any given protein. The first method is particularly suited to identify protein-protein interactions. Two proteins of interest are over-expressed in yeast, one as a fusion protein with the DNA-binding domain of the GAL4 transcription factor, and the other as a fusion with the transcription-activating domain of the same factor. If the two proteins of interest interact in the yeast cell, the activating domain comes into vicinity of the DNA-binding domain, and the respective promoters are turned on, which can be monitored using a suitable reporter gene driven by a GAL4-dependent promoter. This “yeast two-hybrid” method has had great success in identifying new protein-protein interactions. Its limitations come from the fact that the proteins are over-expressed, usually in a heterologous system. The two proteins are therefore torn out of their natural context, i.e., native complexes. Therefore, the method only indicates that two proteins are able to interact with each other, not whether this interaction occurs under physiological conditions. Consequently, a lot of false-positives are identified.

The second method therefore aims at purifying native complexes and to identify their components using standard proteomic approaches. This was done for a long time by expressing the protein in its native system as a fusion protein with an “affinity” tag, for which simple and efficient purification procedures have been developed. Such tags include the histidine (His) tag, the glutathione-S-transferase (GST) tag, the maltose-binding tag, the calmoduline-binding peptide (CaMB-tag), or the strepavidin-binding peptide (strep-tag). Purification of a protein from eukaryotic extracts by any of these affinity tags, however, does not yield a complex that is pure enough to identify its components by proteomic methods. Therefore, it was a major breakthrough to employ two affinity tags in a “tandem affinity purification”: combining two affinity steps routinely yields preparations of sufficient purity. This method has been used in a large-scale screen to characterise the complexes of every known protein in the baker's yeast S. cerevisiae. The drawback of this method comes from the fact that the protein has to be tagged at the genetic level. The yeast is very accessible for such an approach, as exogenous, tagged genes are easily introduced into this organism, and sufficient biological material can be grown in short time. Higher eukaryotes are not so easy to handle. Furthermore, the endogenous, untagged version of the protein is preferentially incorporated into many native complexes, and it is therefore advisable to substitute the endogenous gene for the tagged version, rather than inserting an additional, tagged copy. Again, this is easily done in yeast but much more demanding in higher eukaryotes.

A powerful agent for the highly specific binding of particular proteins has been available for decades: artificially raised antibodies. Indeed, antibodies for many mammalian proteins are commercially available.

Antibodies usually recognise and bind proteins with exceptionally high specificity and are also suited to purify the antigen protein and associated macromolecules. However, they are not normally useful to obtain, in a simple one-step procedure, the high purity required for proteomic analysis, because they actually bind their target proteins too tightly and therefore cannot be eluted under mild conditions. Only few antibodies bind their target reversibly, and not all of these can be used to purify native complexes free of excessive background contamination. Thus, only very few antibodies are useful in this kind of purification and this strategy therefore is not generally applicable.

### DESCRIPTION OF THE INVENTION

In this invention, is described an artificial adapter protein that combines an antibody-binding activity with two affinity tags and its use in isolation of antibody-antigen complexes. Using this adapter protein, complexes can be obtained at good yield and in the high purity necessary for the identification of all biological macromolecules that are associated with the antigen.

One application of the artificial adapter protein according to the invention is the purification of complexes of macromolecular complexes including, but not limited to, complexes containing proteins and/or nucleic acids, following the subsequent steps:

(1) immobilising the antibody via the adapter protein on a suitable resin (solid support),

(2) passing over the resin a preparation of proteins and/or nucleic acids that contains the respective antigen,

(3) recovering the antigen complexed to the antibody-adapter complex by affinity chromatography, using the affinity tag(s) chosen for the adapter protein, and

(4) identification of co-purifying proteins and/or nucleic acids by standard molecular biology / proteomic techniques.

Another application of the invention can be the identification of antigens that react with a given antigen population for example, but not limited to, in screening of autoimmune patients. This application follows essentially the steps of the first application. In this application, it may be necessary to suppress interactions of the antigen with other macromolecules by, for example, raising the salt concentration during the purification or by adding chaotropic agents (urea, weakly denaturing detergents).

Another application of the invention can be the purification of cells that express a given antigen on their surfaces for purposes that may include, but are not limited to, cloning of the respective cells. This procedure follows the subsequent steps:

(1) immobilising the antibody via the adapter protein on a suitable resin

(2) passing a suspension of a heterogeneous mixture of cells in suspension over the resin,

(3) recovering the cells expressing the antigen by affinity chromatography, which is defined by the affinity tag(s) chosen for the adapter protein,

(4) culturing the affinity-selected cells for analysis and/or expansion.

To apply the method of this invention, it is necessary to have access to a suitable antibody. Suitable antibodies are any kind of antibody that recognise and bind specifically the macromolecule of interest in its native state. The native state of a macromolecule is the folding in which the respective molecule is found in its natural, physiological environment or in which the macromolecule exerts its function. Antibodies can be produced as polyclonals in rabbits or any other vertebrate animal; as monoclonals, for example in mouse hybridoma cell cultures; or by any other convenient method, as long as the respective antibodies can be bound by the adapter molecule. If the scope of the purification is the isolation and characterisation of biological macromolecular complexes, polyclonal antibodies are preferably, but not necessarily, enriched by affinity-selection using standard procedures. For many proteins, antibodies that are suitable for the method of this invention are commercially available.

The main object of this invention is a biological macromolecule that binds the antibody and that can be purified by one or more steps of affinity purification (“adapter”). It has therefore a modular structure: one part consists of an antibody-binding domain. This domain is preferably but not necessarily a protein, preferably the well-studied protein A of Staphylococcus aureus, or one or more copies of its IgG-binding domain. Alternatively, it is also possible to use protein G of Staphylococcus aureus or fragments thereof, a secondary antibody directed against the specific antibody, a recombinant fragment of the secondary antibody, or any other biological macromolecule that specifically binds to antibodies of the kind selected for the purification protocol.

This antibody-binding module is coupled to one or more affinity tags. An affinity tag in this sense is any molecule that can be conjugated with the antibody-binding module and that allows rapid purification of the tagged protein in its native state. In a preferred embodiment of the invention, the affinity tags are peptides or protein domains that are expressed together with the IgG-binding module as one polypeptide. Such affinity tags include, but are not limited to, the His tag that binds to immobilised nickel ions (patent), the strep tag that binds to immobilised, modified streptavidin (patent?), or a calmodulin-binding peptide which binds to immobilised calmodulin (patent). Alternatively, the tag can be any molecule or macromolecule conjugated chemically to the antibody-binding module, as long as there is a convenient purification scheme to affinity-select this (macro)molecule, or a combination of a peptide affinity tag and a chemically conjugated tag.

Another aspect of this invention is the piece of nucleic acid that encodes the adapter protein, preferably but not necessarily a plasmid DNA. If this nucleic acid is a plasmid with the necessary regulatory elements from bacteria, the adapter protein can be expressed in E. coli using standard procedures and purified through one or more of the tags that have been added to the IgG-binding domain or by affinity selection using immobilised antibodies. Alternatively, the adapter protein can be produced by any other convenient method, including but not limited to expression in different host organisms, translation in vitro, or chemical synthesis.

In one embodiment of the invention, the purification is used to isolate and characterise biological macromolecular complexes that are centered on the protein of interest. Complexes are associations of several macromolecules that are stable enough to withstand the affinity purification. Thereby, the method identifies other biological macromolecules, usually but not necessarily proteins and/or nucleic acids, that stably associate with the protein of interest. For the purification according to the protocol, the macromolecular complex has to be soluble or can be solubilised without disintegrating. To identify physiologically relevant complexes, they can be isolated by extracting biological material with physiological buffers. Such extracts can be prepared from entire organisms, isolated tissues, or cultured cells according to standard procedures. It should be noted that the use of denaturing agents during extract preparation compromises the stability of complexes and thus limits the possibilities to recover associated factors. In the same way, sub-cellular organelles or even entire cells can be purified and their content analysed as long as they present the protein of interest on the respective membrane and they enter into the pores of the solid support chosen for the purification.

In another embodiment of the invention, molecule-molecule interactions can be tested even if one of the possible partners tends to aggregate non-specifically and therefore gives a high background in conventional techniques. In this case, preparations of the interaction partners are mixed in vitro and the mixture is then used as starting material to purify stably associated heteromers.

Complexes containing the protein of interest, associated proteins, the antibody and the adapter protein are purified by affinity chromatography using one or more tags added to the adapter. It is important to choose the tag(s) such that the purification can be performed under native conditions, that is, without dissociating the complex. In this way, preparations that are only partially pure can be subjected to a second purification step that uses a second type of tag added to the same adapter molecule. In this way, preparations of extremely high purity are routinely obtained. These preparations contain the adapter molecule, the antibody, the antigen, and any molecules that bind to the antigen, which can be proteins, nucleic acids, or other. These constituents of the complex are identified by standard biological/proteomic or chemical procedures.

In another embodiment of the invention, the method described above is combined with one or more additional purification steps This can include, but is not limited to, methods to separate macromolecular complexes by size, such as gel filtration or density gradient centrifugation. This extra step will separate excess complexes that contain only the antibody and the adapter from the complexes that also contain the antigen and associated molecules. At the same time, it is possible to determine whether several constituents of the antigen complex are present in the same complex or bind the antigen in a mutually exclusive manner.

### EXAMPLE 1

### Purification of Two snRNP-related Complexes From Nuclear Extract of Human HeLa Cells

The adapter protein was, in this case, a fusion of a calmodulin-binding peptide with a streptavidin-binding peptide and two copies of the proteinA IgG-binding domain. 0.2 ml of calmodulin-coated beads were incubated for 30 min at 4° C. with 20 μg of adapter protein in 2 ml of CaM-binding buffer (10 mM Tris-Cl pH 8.0; 150 mM NaCl, 1 mM MgCl₂, 1 mM Imidazol, 2 mM CaCl₂, 0.1% Triton, 10 mM □-ME). The beads were washed three times with 1 ml of CaM-binding buffer, and then antibodies directed against SF3a120 (second lane) or SF3b155 (third lane) were added in 2 ml of CaM-binding buffer. Both antibodies were affinity-purified polyclonals raised in rabbit. As a control, a third batch of adapter protein was immobilised on the same beads and then incubated without antibodies (first lane). The beads were washed as before. For each precipitation, 1 ml of nuclear extract (ca. 5 mg of protein, extracted from approximately 1 gr. of HeLa cells) was diluted with 9 ml CaM-binding buffer and cleared by centrifugation at 10000 g for 15 min. The extract was added to the beads and incubated for 1 hr at 4° C. The beads were washed three times with 5 ml CaM-binding buffer and then eluted with CaM-elution buffer (10 mM Tris-Cl pH 8.0; 150 mM NaCl, 1 mM MgCl₂, 1 mM Imidazol, 20 mM EGTA, 0.1% Triton, 10 mM □-ME) in 5 fractions of 0.2 ml each. Fractions 2 to containing the bulk portion of eluted protein were pooled, diluted to 2 ml with Strep-binding buffer (20 mM Tris-Cl pH 8.0; 150 mM NaCl, 1.5 mM MgCl₂, 0.1% Triton) and applied to 0.2 ml of streptactin-coated beads (Qiagen). The suspensions were incubated for 1 hr. at 4° C., and then the beads were washed three times with Strep-binding buffer. The purified complexes were eluted with Strep-elution buffer (20 mM Tris-Cl pH 8.0; 150 mM NaCl, 1.5 mM MgCl₂, 0.1% Triton, 2 mM Desthiobiotin) in 5 fractions of 0.2 ml each. For analysis, the protein of 10% of the pooled elute from the CaM-beads and of the entire fractions 2 and 3 from the Streptactin beads was recovered by acetone precipitation and fractionated by SDS polyacrylamide electrophoresis. Proteins were stained with SYPRO Ruby. Bands Visible in the pure preparations were cut out, and the respective proteins were identified by mass-spectrometry.

The elution of the first step is shown in the left panel. The adapter protein and the IgG antibodies can be readily identified, but beyond this the high background that is observed also in the negative control (first lane) does not permit the identification of specific bands co-precipitating with the adapter-IgG complex. The elution after the second step is shown in the right panel. Again, the adapter and the antibodies are readily visible. Only few additional bands are detectable and were analysed by mass-spectrometry. The identity of the proteins is indicated in the panel; bands that are not labelled correspond to human keratin, IgGs, adapter protein, or fragments of these proteins. Importantly, all specific proteins correspond to subunits of the SF3a and SF3b complexes (the two complexes interact with each other and thus co-precipitate). This underlines the extraordinary purity of the preparation.

### EXAMPLE 2

### Purification of Two Complexes From Cytoplasmic Extract of Human HeLa Cells

The procedure described above was used to isolate the antigen complexes of the following antibodies: first lane, negative control without antibody. Second lane, rabbit polyclonal, affinity-purified antibodies directed against the human LSm1 protein. Third lane, mouse monoclonal antibody Y12 directed against the Sm snRNP proteins. 4 ml of cyptoplasmic extract (12 mg of protein, extracted from ca. 1 gr. of human HeLa cells) were used.

As before, the eluate of the first purification step shows an excessive background, whereas the second step yields highly pure complexes (right panel). The monoclonal antibody Y12 precipitates the Sm snRNP proteins and the cross-reactive Sm-like proteins (LSm). The additional proteins are all components of the spliceosomal snRNPs U1 (U1-A), U2 (U2-A′), or U5 (102K, 116K, 200K, hPrp8) which associate with the Sm proteins, again underlining the extraordinary purity of the preparation. Only one protein appears in both precipitates and therefore represents a possible contamination. It should be noted, however, that this possible contamination is not at all detected in the negative control. Further, the Sm and LSm proteins are related and LSm proteins partially co-precipitate with Y12. Therefore, the protein in question may also co-purify specifically with both antibodies. The anti-hLSm1 antibodies co-precipitate other hLSm proteins (they form a known complex), and the human homologue of the yeast factor Pat1p.

Pat1p associates with the yeast protein Lsm1p and is involved in the same process. A human homologue has so far not been identified because it is not sufficiently conserved to pick it up by sequence comparison. The fact that this protein co-purifies with hLSm1, however, renders the annotation as human Pat1 unambiguous. This demonstrates that the method described in the patent is useful to identify de novo factors that interact with a protein of interest.

1-14. (canceled)

15. A method to isolate and/or characterize complexes of biological macromolecules or parts thereof, comprising:
(i) immobilizing a selected antibody that recognizes at least one component of a complex of a biological macromolecule using an adapter molecule that combines an antibody-binding activity with at least one affinity tag;
(ii) binding the complex that is centered on an antigen macromolecule to the antibody immobilized in (i); and
(iii) purifying and characterizing the complex attached to the antibody plus an adapter protein by at least one affinity purification step using the at least one affinity tag attached to the adapter molecule.

16. A method to identify antigens of an antibody population comprising:
(i) immobilizing the antibody population using an adapter molecule that combines an antibody-binding activity with at least one affinity tag;
(ii) binding a mixture of possible antigens to the antibody population immobilized in (i); and
(iii) purifying and characterizing selected antigens attached to an antibody of the antibody population plus an adapter protein by at least one affinity purification step using the at least one affinity tag attached to the adapter molecule.

17. A method to isolate cells from a pool of polyclonal/heterogeneous cells comprising:
(i) immobilizing an antibody that recognizes a surface marker that is expressed on selected cells of interest using an adapter molecule that combines an antibody-binding activity with at least one affinity tag;
(ii) binding the cells to the antibody immobilized in (i); and
(iii) purifying the cells expressing the surface marker by at least one affinity purification step using the at least one affinity tag attached to the adapter molecule, wherein the at least one affinity purification step is carried out in the absence of denaturing steps, wherein the selected cells can be cultured and expanded after selection.

18. The method according to one of claims 15 to 17, wherein protein A from Staphylococcus aureus, or fragments of the protein A, is used to bind the antibody.

19. The method according to one of claims 15 to 17, wherein a double copy of a IgG-binding domain of protein A from Staphylococcus aureus is used to immobilize the antibody.

20. The method according to one of claims 15 to 17, wherein at least one of the at least one affinity tag is a Calmodulin-binding peptide.

21. The method according to one of claims 15 to 17, wherein at least one of the at least one affinity tag is a strep-tag peptide.

22. An adapter molecule able to bind antibodies of any type comprising at least one affinity tag.

23. The adapter molecule according to claim 22, further comprising a strep-tag, a Calmodulin-binding peptide and two copies of a IgG binding domain of protein A.

24. Nucleic acid that encodes the adapter molecule according to claim 22 or 23.

25. Organism/bacteria that can express the adapter molecule according to claim 22 or 23.

26. Reagent kit that includes materials necessary to carry out the a method according to one of claims 15 to 17.

27. The reagent kit according to claim 26, comprising the adapter molecule, a nucleic acid encoding the adapter molecule, or an organism/bacteria expressing together with one or more support materials capable of binding a tag added to the adapter molecule and agents to elute the materials from a support.

28. The method according to claim 15, wherein said method identifies molecules that are capable of associating with a selected macromolecule or verifies such an association.
